(19) United States
(12) Patent Application Publication
(10) Pub. No.: US 2022/0370477 A1
(43) Pub. Date: Nov. 24, 2022

(54) Solid Oral Dosage Forms Of Dexamethasone

(71) Applicant: Jubilant Generics Limited, Noida (IN)

(72) Inventors: Mitesh Nagar, Noida (IN); Nilesh Jaiswal, Noida (IN); Indranil Nandi, Yardley, PA (US); Sunil Yadav, Noida (IN); Dinesh Kumar, Noida (IN)

(73) Assignee: Jubilant Generics Limited, Noida (IN)

(21) Appl. No.: 17/742,843

(22) Filed: May 12, 2022

(30) Foreign Application Priority Data
May 14, 2021 (IN) 202111021793

(51) Int. Cl. A61K 31/573; A61K 31/454; A61K 9/00; A61K 9/16; A61K 9/20; A61P 7/00

(52) U.S. Cl. CPC A61K 31/573; A61K 31/454; A61K 9/0053; A61K 9/1694; A61K 9/2018; A61K 9/2027; A61K 9/2059; A61P 7/00

(57) ABSTRACT

The present invention relates to solid oral pharmaceutical compositions comprising dexamethasone or its pharmaceutically acceptable salts or solvates thereof. The present invention also relates to a process for preparing solid oral pharmaceutical compositions comprising dexamethasone or its pharmaceutically acceptable salts or solvates thereof. The preferred drug load in the compositions of the present invention is from about 0.01% to 15% by weight based on the total weight of the composition. The prepared compositions of dexamethasone as per the present invention exhibit desirable technical attributes.

### FIELD OF THE INVENTION

The present invention relates to solid oral pharmaceutical compositions of dexamethasone or its pharmaceutically acceptable salts or solvates. In particular, but without restriction to the particular embodiments hereinafter described in accordance with the best mode of practice, the present invention provides a solid oral pharmaceutical composition comprising dexamethasone or its pharmaceutically acceptable salts or solvates and processes for preparing the same.

### BACKGROUND OF THE INVENTION

Dexamethasone is a synthetic steroidal glucocorticoid, which is used in the treatment of many conditions, including various skin diseases, severe allergies, asthma, chronic obstructive lung disease, rheumatic problems, and along with antibiotics in tuberculosis, to name a few. Dexamethasone is chemically known as 9-fluoro-11β,17,21trihydroxy-16α-methylpregna-1,4-diene-3 ,20-dione. Dexamethasone is practically insoluble in water, sparingly soluble in acetone, ethanol, and methanol, and slightly soluble in dichloromethane and is represented by the following formula as:

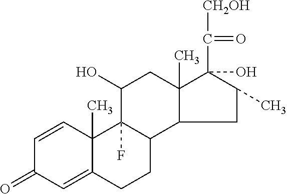

Solid oral dosage forms of dexamethasone are approved in the US market as immediate-release tablets in strengths of 0.25 mg, 0.5 mg, 0.75 mg, 1 mg, 1.5 mg, 2 mg, 4 mg, and 6 mg for the treatment of various diseases such as allergic, dermatologic, endocrine, gastrointestinal, hematologic, renal, respiratory, rheumatic diseases and palliative management of leukemias and lymphomas.

Recently, dexamethasone was approved as HEMADY® by Dexcel Pharma as an immediate-release tablet in a strength of 20 mg. HEMADY® tablet dosage form contains dexamethasone along with inactive ingredients such as corn starch, lactose monohydrate, magnesium stearate, povidone, and sodium starch glycolate.

Dexamethasone is reported to be poorly water-soluble, which leads to poor dissolution and bioavailability. Poor aqueous solubility, flowability, and compressibility of the drug also pose technical challenges to formulation scientists in the development of a suitable formulation with desired technical attributes.

U.S. Pat. Nos. 10,537,585 and 11,304,961 assigned to Dexcel Pharma discloses high dose dexamethasone compositions. The said patent further discloses a pharmaceutical composition comprising dexamethasone in an amount of from 15 to 25 weight percent along with other excipients such as a filler, a binder, a disintegrant, and a lubricant. These patents disclose the use of binders such as starch and polyvinylpyrrolidone in tablet composition in the range of 10-30% by weight.

US Patent Publication No. 2006/0034915 assigned to Constant Research & Development discloses a tablet composition, wherein dexamethasone is present in an amount of from 2 to 0.02% w/w and a viscosity increasing excipient (starch) from 0.5% to 5% w/w. The said patent publication highlights that dexamethasone is a potent corticosteroid, a small amount of the drug is needed in oral dosage form, i.e., 0.25 mg in 300 mg tablet (0.083%).

US Patent Publication No. 20210275546 assigned to Jubilant Pharma Holdings Inc. discloses high drug load solid oral pharmaceutical compositions comprising dexamethasone, wherein dexamethasone is present in an amount of from about 26% to 50% by weight based on the total weight of the composition.

Thus, there is a need for an alternate dosage form of dexamethasone with desirable technical formulation attributes such as disintegration, dissolution, improved flow characteristics such as bulk density, tapped density, Hausner ratio, compressibility index, content uniformity, stability, and bioequivalence, and which can be manufactured by a simple, reproducible and commercially viable process at lab scale as well as at industrial scale.

The present inventors have developed an alternate dosage form of dexamethasone, which offers desirable formulation characteristics like disintegration, dissolution, improved flow characteristics such as bulk density, tapped density, Hausner ratio, compressibility index, content uniformity, stability, and bioequivalence which is comparable to the commercially available counterpart (HEMADY® Tablets). Further, the process employed in the manufacture of the test tablet dosage form of dexamethasone is scalable and therefore feasible for industrial production.

### OBJECT AND SUMMARY OF THE INVENTION

It is a principal object of the present invention to provide a solid oral pharmaceutical composition comprising dexamethasone or its pharmaceutically acceptable salts or solvates and the process for preparing such compositions.

The present invention also relates to a solid oral pharmaceutical composition comprising dexamethasone and one or more pharmaceutically acceptable excipients and processes for preparing such compositions.

The present invention also relates to solid oral pharmaceutical compositions comprising dexamethasone and one or more pharmaceutically acceptable excipients, like diluent, disintegrant, lubricant, glidant, and surfactant.

The present invention further relates to solid oral pharmaceutical compositions comprising dexamethasone and one or more pharmaceutically acceptable excipients, which exhibit desired pharmaceutical technical attributes such as disintegration, dissolution, improved flow characteristics such as bulk density, tapped density, Hausner ratio, compressibility index, content uniformity, stability, and bioequivalence.

The present invention also relates to the use of solid oral pharmaceutical compositions of the present invention in the manufacture of medicaments for treating multiple myeloma in combination with at least one anti-cancer drug, allergy states, dermatological diseases, endocrine disorders, gastrointestinal diseases, hematological diseases, neoplastic diseases, nervous system disorders, ophthalmic diseases, renal diseases, respiratory diseases, rheumatic diseases, trichinosis, and tuberculous meningitis.

### DETAILED DESCRIPTION

The present invention can be more readily understood by reading the following detailed description of the invention and study of the included examples.

As used herein, the term “composition”, as in pharmaceutical composition, is intended to encompass a drug product comprising dexamethasone or its pharmaceutically acceptable salts, esters, solvates, polymorphs, enantiomers or mixtures thereof, and other inert ingredient(s) (pharmaceutically acceptable excipients). Such pharmaceutical compositions are synonymous with “formulation” and “dosage form”. Preferably, the pharmaceutical composition refers to tablets and capsules. More preferably, the pharmaceutical composition refers to immediate-release oral tablets, which may be uncoated or film-coated. In one embodiment, the pharmaceutical composition refers to granule, powder, sprinkle, granule/powder for solution or suspension (POS) dosage form. The powder or granule composition of the present invention can be ingested directly by the patient i.e. into the patient's mouth, or sprinkled onto food before ingestion, or reconstituted with suitable liquid before administration. More preferably, the pharmaceutical composition refers to uncoated immediate-release oral tablets.

The term “immediate release,” as used herein, implies that dexamethasone is released from the composition in an immediate release fashion such that the composition release more than 70% release of the dexamethasone in 45 minutes or less. In an embodiment, the composition release more than 70% release of the dexamethasone in 45 minutes or less across all physiological pH ranges such as 1.2, 4.5, and 6.8.

As used herein, the term “dexamethasone” is used in a broad sense to include not only “dexamethasone” per se (free base) but also its pharmaceutically acceptable salts, solvates, hydrates, enantiomers, derivatives, isomers, metabolites, polymorphs, prodrugs thereof. Polymorph may refer to various crystalline and amorphous forms of dexamethasone, which can be characterized by methods such as melting point, X-ray diffraction pattern, Raman spectra, IR spectra, or any other method known in the art. Suitable salts of dexamethasone include but are not limited to, acid addition salts and base addition salts such as sodium, potassium, zinc, magnesium, hydrochloride, hydrobromide, sulfate, phosphate, nitrate, and like, amino acids salts such as glycine, lysine, arginine, or any other known salt in the art. In a preferred embodiment, dexamethasone is present in free base form.

The composition as per the present invention includes the amount of drug from about 0.01% to about 97% by weight of dexamethasone based on the total weight of the composition. Preferably, the solid oral pharmaceutical composition as per the present invention comprises from about 0.01% to about 50%, about 0.01% to about 40% of dexamethasone. More preferably, the solid oral pharmaceutical composition as per the present invention comprises from about 0.01% to about 15% of dexamethasone. The solid oral pharmaceutical composition as per the present invention comprises from about 5% to about 15% of dexamethasone. More preferably, solid oral pharmaceutical composition as per the present invention comprises from about 10% to about 13.5% of dexamethasone. Preferably, the solid oral pharmaceutical composition as per the present invention does not comprise less than 9% of dexamethasone.

The term “excipient” means a pharmacologically inactive component such as a diluent, binder, disintegrant, glidant, surfactant, wetting agent, lubricant, solid carrier, solubilizer, stabilizer, sweetener, flavoring agent, coloring agent, and the like.

“Substantially free” or “free” as used herein refers to the pharmaceutical composition of dexamethasone, which is devoid of said element/component/excipient or may contain less than 10% or less than 5% or less than 2% or preferably less than 1% of the said element/component/excipient. The term “starch” includes but is not limited to corn starch, pregelatinized starch, sodium carboxymethyl starch, microcrystalline starch, or other starch derivatives thereof.

As used herein, the term “intra-granular” (part/phase/portion) refers to the components of formulation of the present invention that are within granules. As used herein, the term “extra-granular” (part/phase/portion) refers to those components of the formulation of the present invention that are outside the granules.

Unless otherwise stated the weight percentages expressed herein are based on the final weight of the composition or formulation. As used herein, the term “about” means ±approximately 20% of the indicated value, such that “about 10 percent” indicates approximately 8 to 12 percent.

The compositions prepared by the processes as per the present invention are tested for physical parameters such as weight variation, hardness, thickness, bulk density, tapped density, assay, content uniformity, disintegration test, friability, compressibility index, Hausner ratio, disintegration, and dissolution. Several devices can be used to test tablet hardness such as a Monsanto tester, a Strong-Cobb tester, a Pfizer tester, an Erweka tester, a Schleuniger tester, etc. In some embodiments, the tablet according to the present invention exhibits a target hardness of about 30 N to about 150 N. In an embodiment, the hardness of the tablet according to the present invention is about 150 N or less, e.g., 140 N or less, 130 N or less, 120 N or less, 110 N or less, 100 N or less, 90 N or less, 80 N or less. In some embodiments, the tablet according to the present invention exhibits a target hardness of about 30 N to about 110 N.

Friability can be determined using a Roche friabilator for 100 revolutions at 25 RPM. In some embodiments, the tablet according to the present invention exhibits friability of about 1% or less. In an embodiment, the friability of the tablet according to the present invention is about 1% or less, e.g., 0.9% or less, 0.8% or less, 0.7% or less, 0.6% or less, 0.5% or less, 0.4% or less, 0.3% or less, 0.2% or less. In a preferred embodiment, the friability of the tablet according to the present invention is about 0.5% or less.

Disintegration time testing for tablets can be performed in a USP tablet disintegration tester wherein a tablet is placed in a basket, which moves upward and downward in a 1-liter beaker of water at 37° C.

The term “stable” refers to the compositions of the present invention in which the amount of active ingredient of a formulation does not deviate from the initial amount by more than the values given in the specification or the guidelines of the common Pharmacopoeias or loss in active ingredient is less than 50% of the initial content after being stored for at least 1 month, preferably for at least 2 months, preferably for at least 3 months, more preferably for at least 6 months, more preferably for at least 12 months or more preferably for at least 24 months. The stability of the solid oral composition may be evaluated at “long term” conditions 25° C./60% RH (relative humidity), at “intermediate conditions” 30° C./65% RH, at “accelerated conditions” 40° C./75% RH, in the final container either measured as the loss in the content of active ingredient. Stability testing may be conducted according to the current guidelines by ICH (International Council for Harmonisation) and USFDA (United States Food and Drug Administration).

The pharmaceutical compositions of the present invention comprise about 0.1 to about 50 mg of dexamethasone, preferably about 2 to about 40 mg of dexamethasone, more preferably about 20 mg of dexamethasone. In another embodiment, the composition of the present invention includes dexamethasone as the sole active ingredient. In another embodiment, the composition comprises 20 mg of dexamethasone thereof.

In another embodiment, the pharmaceutical composition of the present invention includes particle sizes of dexamethasone, having a particle size distribution such that D₉₀ is less than about 200 μm, D₅₀ is less than about 100 μm and D₁₀ is less than about 50 μm. The particle size of dexamethasone can be measured by suitable techniques such as Laser light scattering (e.g. Malvern Light Scattering), Coulter counter, microscopy, and any other technique known in the art. In an embodiment, the D₉₀ according to the present invention is present in about 200 μm or less, e.g., 180 μm or less, 140 μm or less, 100 μm or less, 80 μm or less, 60 μm or less, 40 μm or less, 20 μm or less, 10 μm or less. In an embodiment, the D₅₀ according to the present invention is present in about 100 μm or less, e.g., 80 μm or less, 60 μm or less, 50 μm or less, 40 μm or less, 20 μm or less, 10 μm or less. In an embodiment, the D₁₀ according to the present invention is present is about 50 μm or less, e.g., 40 μm or less, 30 μm or less, 20 μm or less, 10 μm or less, 5 μm or less. In a preferred embodiment, dexamethasone has a particle size distribution such that D₉₀ is about 10 μm or less, D₅₀ is about 7 μm or less and D₁₀ is about 2 μm or less.

In another embodiment of the invention, the solid oral pharmaceutical composition comprising dexamethasone is prepared by a wet or dry process. The wet and dry processes include, but are not limited to, wet granulation, dry granulation, dry blending, dry mixing, and direct compression. Any pharmaceutically acceptable granulating agent can be used for wet granulation. Preferably, granulating solvents include, but are not limited to, water, esters such as ethyl acetate, ketones such as acetone, alcohols such as methanol, ethanol, isopropanol, butanol, dichloromethane, chloroform, dimethylacetamide (DMA), dimethyl sulfoxide (DMSO), ether, diethyl ether and combinations thereof. Preferably, the granulating solvent used during wet granulation is water. In another embodiment of the invention, wet granulation can be performed using a rapid mixer granulator, a fluid bed granulator, a planetary mixer, and the like; dry blending can be performed using V-blender or key blender, and dry granulation can be performed using a roller compacter or slugging techniques or by any other method known in the art. The granules obtained as per the present invention are dried at a suitable temperature in a suitable drying apparatus until the desired loss on drying value is obtained. In an embodiment, the Loss On Drying (LOD) of the granulate and/or tablet composition is in the range of about 1% to about 8.5%, preferably about 1% to about 7%, preferably from about 1% to about 5%. The water content of the tablets can be measured using the loss on drying method or the Karl Fischer method, which are well known to those skilled in the art.

The present invention relates to a solid oral pharmaceutical composition comprising dexamethasone or its pharmaceutically acceptable salts or solvates thereof and processes for preparing such compositions.

In another embodiment of the invention, there is provided a solid oral pharmaceutical composition comprising dexamethasone and one or more pharmaceutically acceptable excipients, like diluent, binder, disintegrant, lubricant, glidant, and surfactant.

In another embodiment of the invention, there is provided a solid oral pharmaceutical composition comprising dexamethasone in an amount of from about 5% to about 15% by weight and one or more pharmaceutically acceptable excipients, like diluent, disintegrant, lubricant, glidant, and surfactant.

In another embodiment of the invention, the amount of dexamethasone may vary from about 5% to about 15% by weight, preferably, from about 10% to about 14% by weight, more preferably from about 10% to about 13.5% or 10% to about 12.5% by weight, based on the total weight of the composition.

In a preferred embodiment, the immediate-release solid oral pharmaceutical tablet composition does not comprise less than 3% by weight of dexamethasone. In a more preferred embodiment, the immediate-release solid oral pharmaceutical tablet composition comprises 12.5% by weight of dexamethasone.

In another embodiment, the present invention includes a solid oral pharmaceutical composition of dexamethasone comprising from about 5% to about 15% by weight of dexamethasone, preferably from about 10% to about 14% by weight of dexamethasone based on the total weight of the composition, wherein the composition is substantially free of any starch excipient. In a further embodiment, the composition as per the present invention is devoid of any starch excipient. In a further embodiment, the composition as per the present invention is free of lactose.

In another embodiment, the composition is substantially free of other undesirable polymorphic forms.

In another embodiment of the invention, there is provided a solid oral pharmaceutical composition comprising dexamethasone and at least one or more pharmaceutically acceptable excipients including a diluent, disintegrant, glidant, surfactant, and lubricant, wherein the said composition is substantially free of a binder. In another preferred embodiment of the invention, the composition is completely free of a binder.

In another embodiment of the invention, there is provided a solid oral pharmaceutical composition comprising dexamethasone and at least one or more pharmaceutically acceptable excipients including a diluent, disintegrant, glidant, surfactant, and lubricant, wherein the composition is free of any starch excipient as a dry binder in the intra-granular part of the composition.

In another embodiment of the invention, there is provided a solid oral pharmaceutical composition comprising dexamethasone and at least one or more pharmaceutically acceptable excipients including a diluent, glidant, surfactant, and lubricant, wherein the composition is substantially free of disintegrant. In another embodiment of the invention, the composition is completely free of disintegrant.

In another embodiment of the invention, there is provided a solid oral pharmaceutical composition comprising dexamethasone and at least one or more pharmaceutically acceptable excipients including a diluent, disintegrant, glidant, surfactant, and a lubricant, wherein the disintegrant is present either in the intra-granular or in the extra-granular part of the composition. In another embodiment, where the disintegrant is present in the intra-granular part of the composition then the extra-granular part of the composition is free of disintegrant. In still another embodiment, where the disintegrant is present in the extra-granular part of the composition then the intra-granular part of the composition is free of disintegrant. In another embodiment, the disintegrant is essentially present in both intra-granular as well as extra-granular portions of the composition in equal proportion.

In another embodiment of the invention, there is provided a solid oral pharmaceutical composition comprising: i) about 0.01% to about 15% by weight of dexamethasone; ii) from about 0 to about 95% by weight of one or more diluents; iii) from about 0 to about 30% by weight of one or more binders; iv) from about 0 to about 60% by weight of one or more disintegrants; v) from about 0 to about 20% by weight of one or more surfactants; vi) from about 0 to about 10% by weight of one or more glidants, and vii) from about 0.1% to about 5% by weight of one or more lubricants.

In another embodiment of the invention, there is provided a solid oral pharmaceutical composition comprising: i) about 10% to about 14% by weight of dexamethasone; ii) from about 0 to about 85% by weight of one or more diluents; iii) from about 0 to about 30% by weight of one or more binders; iv) from about 0 to about 20% by weight of one or more disintegrants, and v) from about 0.1% to about 5% by weight of one or more lubricants. In another embodiment of the invention, the composition is free of binder.

In another embodiment of the invention, there is provided a solid oral pharmaceutical composition comprising: i) about 10% to about 14% by weight of dexamethasone; ii) from about 50% to about 85% by weight of one or more diluents; iii) from about 0.01% to about 15% by weight of one or more disintegrants; and iv) from about 0.1% to about 5% by weight of one or more lubricants, wherein the composition is free of binder.

In another embodiment of the invention, there is provided a solid oral pharmaceutical composition comprising: i) about 10% to about 14% by weight of dexamethasone; ii) from about 50% to about 85% by weight of one or more diluents; iii) from about 0.01% to about 15% by weight of one or more disintegrants; iv) from about 0.01% to about 5% by weight of one or more glidant; and v) from about 0.1% to about 5% by weight of one or more lubricants, wherein the composition is free of binder.

In another embodiment of the invention, there is provided a solid oral pharmaceutical composition comprising: i) about 10% to about 13.5% by weight of dexamethasone; ii) from about 50% to about 85% by weight of one or more diluents; iii) from about 0.01% to about 15% by weight of one or more disintegrants, and iv) from about 0.1% to about 5% by weight of one or more lubricants, wherein the composition is free of binder.

In another embodiment of the invention, there is provided a solid oral pharmaceutical composition comprising: i) about 10% to about 14% by weight of dexamethasone; ii) from about 50% to about 85% by weight of one or more diluents; iii) from about 0.01% to about 10% by weight of one or more binders; iv) from about 0.01% to about 5% by weight of one or more glidants; v) from about 0.01% to about 5% by weight of one or more surfactants; and vi) from about 0.1% to about 5% by weight of one or more lubricants, wherein the composition is free of disintegrant.

In another embodiment of the invention, there is provided an immediate-release solid oral pharmaceutical tablet composition, comprising: a) about 9% to about 13.5% by weight of dexamethasone; b) from about 35% to about 90% by weight of one or more diluents selected from the group consisting of lactose, mannitol, microcrystalline cellulose, silicified microcrystalline cellulose, dicalcium phosphate, and combinations thereof; c) from about 0.01% to about 50% by weight of one or more disintegrants selected from the group consisting of croscarmellose sodium, crospovidone, sodium starch glycolate, low substituted hydroxypropyl cellulose, microcrystalline cellulose, pregelatinized starch and combinations thereof; and d) from about 0.01% to about 2% by weight of one or more lubricants selected from the group consisting of stearic acid, hydrogenated vegetable oil, glyceryl behenate, magnesium stearate, and combinations thereof; wherein the tablet composition is free of any binder. In another embodiment of the invention, the immediate-release solid oral pharmaceutical tablet composition further comprises from about 0.01% to about 1% by weight of one or more glidants selected from the group consisting of calcium silicate, magnesium silicate, magnesium trisilicate, talc, colloidal silicon dioxide, and combinations thereof. In another embodiment of the invention, the immediate-release solid oral pharmaceutical tablet composition further comprises from about 0.01% to about 5% by weight of one or more surfactants selected from the group consisting of sodium lauryl sulphate; sorbitan fatty acid esters; polyoxyethylene sorbitan fatty acid esters; polyoxyethylene alkyl ethers; polyoxyethylene castor oil; polyoxyethylene- polyoxypropylene block copolymers; magnesium alumino-meta silicate and combinations thereof.

In another embodiment of the invention, there is provided an immediate-release solid oral pharmaceutical uncoated tablet composition, comprising: a) dexamethasone present from about 11.25% to 13.5% by weight; b) from about 35% to about 90% by weight of one or more diluents selected from the group consisting of lactose, mannitol, microcrystalline cellulose, and combinations thereof; c) from about 0.01% to about 50% by weight of one or more disintegrants selected from the group consisting of croscarmellose sodium, sodium starch glycolate, pregelatinized starch, and combinations thereof; d) from about 0.01% to about 1% by weight of one or more glidants selected from the group consisting of talc, colloidal silicon dioxide, and combinations thereof; and e) from about 0.01% to about 2% by weight of one or more lubricants selected from the group consisting of stearic acid, magnesium stearate, and combinations thereof. In another embodiment of the invention, the composition is free of any binder selected from the group consisting of hydroxypropyl methylcellulose, povidone, starch, and microcrystalline cellulose, and the composition releases more than 80% of the drug within 30 minutes in 500 ml of 0.1N Hydrochloric acid, using USP I apparatus (Basket) at a temperature of 37±0.5° C. and a rotation speed of 100 revolutions per minute and wherein the composition comprises less than 0.5% of 16α-methyl prednisone impurity.

In another embodiment of the invention, there is provided an immediate-release solid oral pharmaceutical uncoated tablet composition consisting of: a) about 11.25% to 13.12% by weight of dexamethasone; b) about 35% to about 85% by weight of lactose; c) about 2% to 47% by weight of croscarmellose sodium or pregelatinized starch; d) about 0.01% to about 0.5% by weight of colloidal silicon dioxide; and e) about 1% by weight of magnesium stearate; wherein the composition is free of any binder selected from the group consisting of hydroxypropyl methylcellulose, povidone, starch, and microcrystalline cellulose and the composition comprises less than 1% of total impurities selected from the group consisting of 16α-methylprednisone, betamethasone, dexamethasone 7,9-diene, desoximetasone, and dexamethasone acetate.

In another embodiment of the invention, there is provided an immediate-release solid oral pharmaceutical uncoated tablet composition consists of: a) about 12.5% by weight of dexamethasone; b) about 39% by weight of lactose; c) about 47% by weight of pregelatinized starch; d) about 0.25% by weight of colloidal silicon dioxide; and e) about 1% by weight of magnesium stearate.

In another embodiment of the invention, there is provided an immediate-release solid oral pharmaceutical uncoated tablet composition consists of: a) about 12.5% by weight of dexamethasone; b) about 82% by weight of lactose; c) about 2% by weight of croscarmellose sodium; d) about 2% by weight of poloxamer; e) about 0.25% by weight of colloidal silicon dioxide; and f) about 1% by weight of magnesium stearate.

In another embodiment of the invention, the immediate-release solid oral pharmaceutical tablet composition releases more than 80% of drug within 30 minutes in 500 ml of 0.1N Hydrochloric acid, using USP I apparatus (Basket) at a temperature of 37±0.5° C. and a rotation speed of 100 revolutions per minute.

In another embodiment of the invention, the immediate-release solid oral pharmaceutical tablet composition is free from any binder selected from the group consisting of hydroxypropyl methylcellulose, povidone, starch, pregelatinized starch, and microcrystalline cellulose.

In another embodiment of the invention, the dexamethasone and disintegrant are used in the ratio of from about 1:0.01 to 1:10. In another embodiment of the invention, dexamethasone and glidant are used in a ratio of from about 1:0.01 to 1:2. In another embodiment of the invention, the dexamethasone and disintegrant are used in the ratio of from about 1:0.1 to 1:4.

In another embodiment of the invention, there is provided a solid oral pharmaceutical composition comprising dexamethasone prepared by wet granulation, extrusion-spheronization, dry granulation, dry blending, dry mixing, or direct compression process.

In another embodiment of the invention, there is provided a solid oral pharmaceutical composition comprising dexamethasone, the composition prepared in a process comprising the steps of: a) sifting the accurately weighed quantities of active agent and one or more pharmaceutically acceptable excipient(s) separately through a suitable sieve; b) mixing of sifted active agent and excipient(s) using a dry mixer; c) lubricating the sifted blend of step b), and d) preparing as granule dosage form or compressing the lubricated blend into tablets.

In another embodiment of the invention, the immediate-release solid oral pharmaceutical uncoated tablet composition is prepared by the following process: a) blending a mixture of dexamethasone and at least one pharmaceutically acceptable excipient; b) optionally compacting the blended material using roller compactor; c) optionally milling the compacted material; d) mixing the blend from step a) or milled material from step c) and lubricating with a suitable lubricant; and e) compressing the lubricated blend from step d) into tablets with suitable tooling.

In another embodiment of the invention, there is provided a solid oral pharmaceutical composition comprising dexamethasone, the composition is prepared in a process comprising the steps of: a) blending a mixture of dexamethasone and at least one pharmaceutically acceptable excipient including a diluent, binder, surfactant and/or a disintegrant; b) compacting the blended material using roller compactor; c) optionally milling the compacted material; d) blend from step b) or addition of milled material from step c) with glidant and lubricating with a suitable lubricant; and e) preparing as granule dosage form or compressing into tablets with suitable tooling.

In another embodiment of the invention, there is provided a solid oral pharmaceutical composition comprising dexamethasone and one or more pharmaceutically acceptable excipients, which exhibited desired pharmaceutical technical attributes such as disintegration, dissolution, improved flow characteristics such as bulk density, tapped density, Hausner ratio, compressibility index, stability, and bioequivalence that is comparable to the commercially available counterpart (HEMADY® Tablets).

The present inventors have surprisingly found that the use of glidant (such as talc, colloidal silicon dioxide, etc.) in specific content and/or in a specific ratio to drug and/or disintegrant was able to overcome multiple formulation challenges such as capping, picking & sticking and were able to develop tablet dosage form with all desired technical attributes. The present inventors also observed that although the wet granulation process was easy to adopt, however, fails to achieve the desired dissolution profile as required for an immediate release tablet which can release 80%-90% drug in 10-15 minutes. The present inventors have surprisingly achieved all desired technical formulation attributes using a direct compression process.

Bioequivalence study: As per the USFDA guideline titled “Bioavailability and Bioequivalence Studies for Orally Administered Drug Products—General Considerations”, Bioequivalence is defined as: “the absence of a significant difference in the rate and extent to which the active ingredient or active moiety in pharmaceutical equivalents or pharmaceutical alternatives becomes available at the site of drug action when administered at the same molar dose under similar conditions in an appropriately designed study.”

In accordance with one embodiment of the present invention, there is provided an immediate-release solid oral pharmaceutical tablet composition, wherein the composition is bioequivalent to the marketed HEMADY® Tablets 20 mg.

In another embodiment, the present invention provides immediate-release solid oral pharmaceutical tablet composition, which is bioequivalent to HEMADY® Tablets 20 mg in a bioavailability study in humans. In another embodiment, the bioavailability study is conducted in humans under fasted and/or fed conditions. In one embodiment, the compositions show comparative pharmacokinetics exposure in terms of point estimates which fall between 80%-125%. In one embodiment, the pharmaceutical compositions when administered to a human is bioequivalent within the 80-125% confidence interval and/or with a statistical power of at least 80% to a 20 mg tablet of dexamethasone in a bioavailability study in humans.

Various useful fillers or diluents include, but are not limited to microcrystalline cellulose (“MCC”), sodium alginate, silicified MCC (e.g., PROSOLV™), microfine cellulose, lactitol, cellulose acetate, kaolin, glucose, lactose, maltose, fructose, sucrose, trehalose, starch, pregelatinized starch, mannitol, xylitol, maltitol, sorbitol, dextrates, dextrin, maltodextrin, compressible sugar, confectioner's sugar, dextrose, polydextrose, simethicone, calcium carbonate, calcium sulfate, calcium phosphate, dibasic calcium phosphate dihydrate, tribasic calcium phosphate, magnesium carbonate, magnesium oxide, isomalt, and mixtures thereof. The amount of diluent according to the present invention ranges from 0 to about 95% by weight of the composition. In an embodiment, the diluent according to the present invention is present in an amount of about 90% or less, 80% or less, e.g. 70% or less, 60% or less, 50% or less, 40% or less, 30% or less, 20% or less.

Various useful binders include, but are not limited to acacia, guar gum, xanthan gum, alginic acid, sodium alginate, dextrin, carbomer, maltodextrin, methylcellulose, ethylcellulose, hydroxyethyl cellulose, hydroxypropyl cellulose (HPC) (e.g., KLUCEL®), hydroxypropyl methylcellulose (HPMC) (e.g., METHOCEL®), hydroxyethyl methylcellulose, carboxymethyl cellulose sodium, cottonseed oil, povidone (various grades of KOLLIDON®, PLASDONE®), ceratonia, dextrose, polydextrose, starch, gelatin, pregelatinized starch, hydrogenated vegetable oil type I, maltodextrin, microcrystalline cellulose, polyethylene oxide, polymethacrylates and mixtures thereof. The amount of binder according to the present invention ranges from 0 to about 50% by weight of the composition.

Various useful disintegrants and/or super-disintegrants include, but are not limited to croscarmellose sodium, carboxymethyl cellulose sodium, carboxymethyl cellulose calcium, povidone, crospovidone, polacriilin potassium, sodium starch glycolate, alginic acid, sodium alginate, calcium phosphate tribasic, docusate sodium, guar gum, low substituted hydroxypropyl cellulose (L-HPC), magnesium aluminum silicate, methylcellulose, microcrystalline cellulose, silicified microcrystalline cellulose, starch, pregelatinized starch and/or combinations thereof. The disintegrant according to the present invention can be used in intra-granular part or extra-granular or in both parts of the composition in any proportion. The amount of disintegrant according to the present invention ranges from 0 to about 50% by weight of the composition. In an embodiment, the disintegrant according to the present invention is present in an amount of about 40% or less, 30% or less, e.g. 20% or less, 10% or less.

Pharmaceutically acceptable lubricants include stearic acid, zinc stearate, sucrose stearate, sodium benzoate, hydrogenated vegetable oil, calcium stearate, adipic acid, glyceryl palmitostearate, glycerine monostearate, medium-chain triglycerides, glyceryl behenate, sodium lauryl sulphate, sodium stearyl fumarate, magnesium lauryl sulphate, magnesium stearate, polyethylene glycol. The amount of lubricant according to the present invention ranges from 0 to about 20% by weight of the composition. Preferably, the amount of lubricant according to the present invention ranges from about 0.1% to about 5% by weight of the composition.

Suitable surfactants according to the present invention are selected from ionic or non-ionic surfactants. Various useful surfactants include, but not limited to, sodium lauryl sulphate, polysorbates (e.g. polysorbate 20, polysorbate 40, polysorbate 60, and polysorbate 80), cetrimide, cetyl alcohol, stearyl alcohol, cetyl stearyl alcohol, cholesterol, polyethylene glycols or its derivatives, polyglycerin fatty acid esters such as decaglyceryl monolaurate and decaglyceryl monomyristate, sorbitan fatty acid esters such as sorbitan monostearate, polyoxyethylene sorbitan fatty acid esters such as polyoxyethylene sorbitan monooleate, polyoxyethylene alkyl ether such as polyoxyethylene lauryl ether, polyoxyethylene castor oil, polyoxyethylene polyoxypropylene block copolymers, and combinations thereof. Commercially available surfactants such as SEPITRAP® 80 or SEPITRAP® 4000 may also be used. SEPITRAP® 80 comprises from about 35% w/w to about 55% w/w of polysorbate 80 and from about 45% w/w to about 65% w/w of magnesium aluminum silicate. The pharmaceutical composition of the present invention may comprise SEPITRAP® 80 in an amount of from about 0.01% w/w to about 20% w/w of the total composition. The amount of surfactant according to the present invention ranges from 0 to about 30% by weight of the composition. More preferably, surfactant according to the present invention is present in an amount of about 0.01% to about 5% by weight of the composition.

Suitable glidants include, but are not limited to, calcium silicate, magnesium silicate, magnesium trisilicate, stearic acid and its derivatives or esters like magnesium stearate, calcium stearate, and sodium stearate and the corresponding esters such as sodium stearyl fumarate, talc, and colloidal silicon dioxide, tribasic calcium phosphate, starch or mixtures thereof. The amount of glidant according to the present invention ranges from 0 to about 20% by weight of the composition. In an embodiment, the glidant according to the present invention is present in an amount of about 20% or less, e.g. 10% or less, 5% or less, 3% or less.

Other carrier materials (such as solid carriers, anti-adherents, solubilizers, stabilizers, colorants, flavors, sweeteners, and preservatives) that are known in the pharmaceutical arts may be included in the composition of the present invention.

The solid oral tablet dosage form prepared by the above process can be subjected to in vitro dissolution evaluation according to Test 711 “Dissolution” in the United States Pharmacopoeia 37, United States Pharmacopoeial Convention, Inc., Rockville, Md., 2014 (“USP”) to determine the rate at which the active substance is released from the dosage form, and the content of the active substance can be determined in solution by high-performance liquid chromatography. When comparing the test and reference products, dissolution profiles should be compared using a similarity factor (f2). The similarity factor is a logarithmic reciprocal square root transformation of the sum of squared error and is a measurement of the similarity in the percent (%) of dissolution between the two curves. Two dissolution profiles are considered similar when the f2 value is equal to or greater than 50.

f2=50·log{[1+(1/n)Σₜ₌₁₋ₙ(Rt−Tt)²]−0.5·100 }

Embodiments of the present invention also relate to a solid oral pharmaceutical composition comprising dexamethasone and one or more pharmaceutically acceptable excipients, wherein the composition exhibits more than 80% of drug release within 30 minutes in 900 ml of 0.1N HCl, using USP II apparatus (Paddle) and/or USP I apparatus (Basket) at a temperature of 37±0.5° C. and a rotation speed of 50-100 revolutions per minute.

In another embodiment, the solid oral pharmaceutical composition of the present invention particularly tablet dosage form may be packaged in HDPE bottles or blister packs. HDPE bottles may optionally contain desiccants. In a preferred embodiment, tablet dosage forms are packaged in HDPE bottles with a desiccant i.e. silica gel.

In accordance with still another embodiment of the present invention, there is provided use of solid oral pharmaceutical compositions of the present invention in the manufacture of a medicament for treating multiple myeloma in combination with at least one anti-cancer drug, allergy states, dermatological diseases, endocrine disorders, gastrointestinal diseases, hematological diseases, neoplastic diseases, nervous system disorders, ophthalmic diseases, renal diseases, respiratory diseases, rheumatic diseases, trichinosis, and tuberculous meningitis.

The following examples are set out to illustrate the invention and do not limit the scope of the present invention.

### EXAMPLES

Dexamethasone tablets were prepared by using the quantitative formula as given in Tables 1, 2, and 3: (Quantity/Tablet (% w/w)).

TABLE 1
Examples 1-3:

| S. No. | Ingredient | Function | 1 | 2 | 3 |
|---|---|---|---|---|---|
| 1. | Dexamethasone | Active Ingredient | 5-15 | 10-15 | 10-13.5 |
| 2. | Lactose/Microcrystalline | Diluent | 0-85 | 30-85 | 50-85 |
| | cellulose/Silicified MCC/ | | | | |
| | Pregelatinized starch | | | | |
| 3. | Povidone/Hypromellose/ | Binder | 0-30 | 0-10 | 0.01-10 |
| | Starch/Microcrystalline | | | | |
| | cellulose | | | | |
| 4. | Sodium starch glycolate/ | Disintegrant | 0-20 | 0-15 | 0.01-10 |
| | Croscarmellose sodium/ | | | | |
| | Crospovidone/Pre-Gelatinized | | | | |
| | starch/Microcrystalline | | | | |
| | cellulose | | | | |
| 5. | Talc/Colloidal Silicon dioxide | Glidant | 0-10 | 0-5 | 0.01-5 |
| 6. | Sodium Lauryl Sulphate/ | Surfactant | 0-20 | 0-10 | 0.01-5 |
| | Poloxamer/Sepitrap-80 | | | | |
| 7. | Magnesium Stearate | Lubricant | 0.1-5 | 0.1-3 | 0.1-3 |

TABLE 2
Examples 4-10:

| S. | | | | | | | | | |
|---|---|---|---|---|---|---|---|---|---|
| No. | Ingredient | Function | 4 | 5 | 6 | 7 | 8 | 9 | 10 |
| 1. | Dexamethasone | Active Ingredient | 13.3 | 12.5 | 13.3 | 12.5 | 12.5 | 12.5 | 12.5 |
| 2. | Lactose | Diluent | 71.7 | — | 46.7 | 72.1 | 72.1 | 72.1 | — |
| 3. | Microcrystalline cellulose | | — | 69.1 | — | — | — | — | — |
| 4. | Silicified MCC | | — | — | — | — | — | — | 75.5 |
| 5. | Pregelatinized starch | | 5.0 | — | 11.7 | — | 7.4 | 9.4 | — |
| 6. | Povidone | Binder | 3.0 | 3.0 | 8.3 | — | 3.0 | 3.0 | — |
| 7. | Hypromellose | | — | — | — | — | — | — | 3.0 |
| 8. | Sodium starch glycolate | Disintegrant | 2.3 | — | 4.0 | — | — | — | 4.0 |
| 9. | Crospovidone | | — | 12.5 | — | 12.5 | — | — | — |
| 10. | Talc | Glidant | 2.3 | 2.5 | — | 2.5 | 2.5 | 2.5 | — |
| 11. | Colloidal Silicon dioxide | | — | — | — | — | — | — | 2.5 |
| 12. | Sodium Lauryl Sulphate | Surfactant | — | — | — | — | 2.0 | — | — |
| 13. | Sepitrap-80 | | — | — | 15.3 | — | — | — | — |
| 14. | Poloxamer | | — | — | — | — | — | — | 2.0 |
| 15. | Magnesium Stearate | Lubricant | 2.3 | 0.4 | 0.7 | 0.4 | 0.5 | 0.5 | 0.5 |

### Preparation Method I

i) Dexamethasone, diluent, and other optional excipients such as disintegrant, binder, and surfactant were sifted through a suitable sieve. ii) The sifted blend of step i) was mixed for a suitable time. iii) Binder and other optional excipients were added in purified water or any other suitable non-aqueous solvent to prepare a binder solution/dispersion. iv) Step ii) blend was granulated using the binder solution/dispersion of step iii). v) The granules of step iv) were dried and optionally sifted and/or milled through a suitable sieve. vi) The granules of step v) were mixed with an optional disintegrant, glidant, and then lubricant was sifted through a suitable sieve and added to granules, and mixed for a suitable time. vii) The blend of step vi) was prepared as granule dosage form or optionally compressed into tablets using suitable punches.

### Preparation Method II

i) Dexamethasone with other optional excipients such as diluent, surfactant, the first portion of disintegrant, and binder were mixed; ii) Binder was dissolved in purified water to prepare a binder solution/dispersion. iii) Blend was granulated using the binder solution/dispersion of step ii); iv) The granules of step iii) were dried and optionally sifted and/or milled through a suitable sieve; v) The remaining second portion of disintegrant was added followed by the addition of lubricant and mixed for a suitable time. vi) The blend of step v) was prepared as granule dosage form or optionally compressed into tablets using suitable punches.

### Preparation Method III

i) The accurately weighed quantities of active agent and other optional excipients such as diluent, disintegrant, binder, and surfactant were sifted through a suitable sieve; ii) The sifted active agent and excipients were mixed using a suitable blender; iii) The sifted blend of step ii) was mixed with glidant and then lubricated, and iv) The lubricated blend was prepared as granule dosage form or optionally compressed into tablets using suitable punches.

### Results

Dissolution: The dissolution profile of tablet compositions prepared using quantitative compositions, was measured in 500 ml of 0.1N Hydrochloric acid, using USP II apparatus (Paddle) at a temperature of 37±0.5° C. and a rotation speed of 50 revolutions per minute. The quantitative composition exhibited more than 70% of drug release within 45 minutes, accordingly dissolution profiles were found acceptable.

TABLE 3
Examples 11-21:

| S. | | | | | | | | | | | | | |
|---|---|---|---|---|---|---|---|---|---|---|---|---|---|
| No. | Ingredient | Function | 11 | 12 | 13 | 14 | 15 | 16 | 17 | 18 | 19 | 20 | 21 |
| 1. | Dexamethasone | Active Ingredient | 11.77 | 11.77 | 11.77 | 11.77 | 11.77 | 11.77 | 11.77 | 12.50 | 12.50 | 12.50 | 12.50 |
| 2. | Lactose | Diluent | 38.99 | 83.64 | 76.39 | 83.64 | 76.39 | 84.62 | 85.52 | 39.25 | 82.00 | 82.00 | 75.00 |
| 3. | Pregelatinized starch | Disintegrant | 48.00 | — | — | — | — | — | — | 47.00 | — | — | — |
| 4. | Croscarmellose sodium | | — | 1.24 | 1.17 | 1.24 | 1.17 | 1.24 | 1.24 | — | 2.00 | 2.00 | 1.25 |
| 5. | Colloidal Silicon dioxide | Glidant | 0.25 | 0.48 | 0.25 | 0.48 | 0.25 | 0.48 | 0.48 | 0.25 | 0.50 | 0.50 | 0.25 |
| 6. | Sodium Lauryl Sulphate | Surfactant | — | — | — | 1.88 | — | — | — | — | — | 2.00 | — |
| 7. | Sepitrap-80 | | — | — | — | — | 9.4 | — | — | — | — | — | 10.00 |
| | (Polysorbate 80 + | | | | | | | | | | | | |
| | Magnesium Alumino- | | | | | | | | | | | | |
| | meta silicate) | | | | | | | | | | | | |
| 8. | Poloxamer | | — | 1.88 | — | — | — | 0.90 | — | — | 2.00 | — | — |
| 9. | Magnesium Stearate | Lubricant | 1.00 | 1.00 | 1.00 | 1.00 | 1.00 | 1.00 | 1.00 | 1.00 | 1.00 | 1.00 | 1.00 |

### Preparation Method I

i) Drug, suitable diluent (such as lactose), surfactant (such as poloxamer), disintegrant (such as croscarmellose sodium), and glidant (silicon dioxide) were mixed in a suitable blender for a suitable time; ii) Blend of step i) was sifted through a suitable sieve; iii) Remaining amount of diluent (such as lactose) was sifted through a suitable sieve; iv) Blend of step ii) and step iii) were mixed in a suitable blender for a suitable time; v) Blend of step iv) was sifted through a suitable sieve; vi) Blend of step v) was mixed in a suitable blender for a suitable time; vii) Suitable lubricant (such as magnesium stearate) was sifted through a suitable sieve and was mixed with blend of step vi); viii) Blend of step vii) was lubricated in a suitable blender for a suitable time; ix) The blend of step viii) was compressed using suitable tooling.

### Preparation Method II

i) Drug, suitable diluent (such as lactose), and glidant (silicon dioxide) were mixed in a suitable blender for a suitable time; ii) Blend of step i) was sifted through a suitable sieve; iii) suitable amount of disintegrant (such as PGS) was sifted through a suitable sieve; iv) Blend of step ii) and step iii) were mixed in a suitable blender for a suitable time; v) Blend of step iv) was sifted through a suitable sieve; vi) Blend of step v) was mixed in a suitable blender for a suitable time; vii) Suitable lubricant (such as magnesium stearate) was sifted through a suitable sieve and was mixed with the blend of step vi); viii) Blend of step vii) was lubricated in a suitable blender for a suitable; ix) The blend of step viii) was compressed using suitable tooling.

### Example 22

The dissolution profile of the tablets dosage form prepared using the quantitative composition as given in Examples 11, 12, 18, and 19 was measured in 500 ml of 0.1N HCl (pH: 1.2) using a USP I apparatus (Basket) at a temperature of 37±0.5° C. and a rotation speed of 100 revolutions per minute and in 500 ml of 0.1N HCl (pH: 1.2) using a USP II apparatus (Paddle) at a temperature of 37±0.5° C. and a rotation speed of 50 revolutions per minute. The dissolution data is provided in Table 4. Assay and water content of prepared compositions were also found in acceptable ranges.

TABLE 4

| Time | % Drug Release | | | |
|---|---|---|---|---|
| Point | Example 11 | Example 12 | Example 18 | Example 19 |
| 10 | 89 | 80 | 71 | 80 |
| 20 | 95 | 92 | 86 | 91 |
| 30 | 96 | 95 | 89 | 95 |
| 45 | 97 | 97 | 90 | 96 |
| 60 | 98 | 98 | 92 | 97 |

### Examples 23

Compositions prepared in Example 18 were subjected to Accelerated stability testing as per the ICH guidelines at a temperature/relative humidity of 40°±2° C./75%±5% RH for 2 months. The Compositions were placed in high-density polyethylene (HDPE) bottle with a silica gel pouch and with induction sealing and analyzed. The prepared dosage form was found to be stable and exhibited the following results (in Table 5):

TABLE 5

| S. | | | |
|---|---|---|---|
| No. | Test | Acceptable Limit | Results |
| 1 | Water content by KF | Not more than 8.5% | 3.91%-5.9% |
| 2 | Assay | 95%-105% | 95.7%-97.9% |
| 3 | Dissolution | Not Less than 80% drug | 88%-90% |
| | | dissolved in 30 minutes | |
| 4 | 16α-methyl | Not more than 0.5% | 0.05%-0.07% |
| | prednisone impurity | | |
| 5 | Total impurities | Not more than 1% | 0.12%-0.18% |

### Example 24

The dexamethasone oral tablets formulation of Example 18 was evaluated for pharmacokinetic parameters in human subjects. The objective of the trial was to characterize the pharmacokinetic exposure and to assess the steady-state comparison of the test formulation (T) with reference formulation (R) [HEMADY® in a healthy adult, human subjects, under fasting and/or fed conditions. The safety of the study subjects was also monitored and evaluated.

### Study Design

The study design was a randomized, open-label, balanced, three-treatment, three-sequence, three-period, three-way crossover, single-dose, oral bioequivalence study. The study was carried out in healthy adults, 36 human subjects, under fasting and fed conditions. Healthy human volunteers who were willing to participate in the study and fulfill the inclusion and exclusion criteria were selected for the study. Blood samples were taken at pre-determined intervals. Pharmacokinetic parameters used to evaluate and compare the relative bioavailability, and therefore bioequivalence, of the two formulations after a single oral dose administration under fasting and fed conditions were Cₘₐₓ, AUC₀₋ₜ, AUC_{0-inf}. Bioequivalence was determined using the 90% confidence interval for the exponential of the difference between the tablet (R) and the test (T). The test met the 80-125% confidence interval limits with a statistical power of at least 80%.

TABLE 6

| | T/R Ratio (%) | T/R Ratio (%) |
|---|---|---|
| Parameter | Fasting | Fed |
| Cₘₐₓ | 92.87 | 88.37 |
| AUC₀₋ₜ | 95.39 | 91.14 |
| AUC_{0-inf} | 95.02 | 90.79 |

### Conclusion

The results indicate that the compositions as per the present invention are bioequivalent to HEMADY® tablets (20 mg). There were no serious adverse events reported during any of the studies.

The compositions disclosed herein can comprise the listed ingredients in the listed amounts, can consist of the listed ingredients, or can consist of the listed ingredients in the listed amounts. In another implementation, the compositions disclosed herein can consist essentially of the listed ingredients and optionally their listed amounts where additional ingredients cannot affect one or more of the pharmaceutical technical attributes such as disintegration, dissolution, and bioequivalence. In another implementation, the additional ingredients cannot affect the flow characteristics such as bulk density, tapped density, Hausner ratio, compressibility index, content uniformity, and stability.

What is claimed:

1. An immediate-release solid oral pharmaceutical tablet composition, comprising:
a) about 9% to about 13.5% by weight of dexamethasone;
b) from about 35% to about 90% by weight of one or more diluents selected from the group consisting of lactose, mannitol, microcrystalline cellulose, silicified microcrystalline cellulose, dicalcium phosphate, and combinations thereof;
c) from about 0.01% to about 50% by weight of one or more disintegrants selected from the group consisting of croscarmellose sodium, crospovidone, sodium starch glycolate, low substituted hydroxypropyl cellulose, microcrystalline cellulose, pregelatinized starch and combinations thereof; and
d) from about 0.01% to about 2% by weight of one or more lubricants selected from the group consisting of stearic acid, hydrogenated vegetable oil, glyceryl behenate, magnesium stearate, and combinations thereof;
wherein the tablet composition is free of any binder.

2. The immediate-release solid oral pharmaceutical tablet composition according to claim 1, further comprises from about 0.01% to about 1% by weight of one or more glidants selected from the group consisting of calcium silicate, magnesium silicate, magnesium trisilicate, talc, colloidal silicon dioxide, and combinations thereof.

3. The immediate-release solid oral pharmaceutical tablet composition according to claim 1, further comprises from about 0.01% to about 5% by weight of one or more surfactants selected from the group consisting of sodium lauryl sulphate;
sorbitan fatty acid esters; polyoxyethylene sorbitan fatty acid esters; polyoxyethylene alkyl ethers; polyoxyethylene castor oil; polyoxyethylene-polyoxypropylene block copolymers; magnesium alumino-meta silicate and combinations thereof.

4. The immediate-release solid oral pharmaceutical tablet composition according to claim 1, wherein the composition comprises 20 mg of dexamethasone.

5. The immediate-release solid oral pharmaceutical tablet composition according to claim 1, wherein the tablet is an uncoated tablet.

6. The immediate-release solid oral pharmaceutical tablet composition according to claim 1, wherein the dexamethasone has a particle size distribution with D90 less than 10 μm.

7. The immediate-release solid oral pharmaceutical tablet composition according to claim 1, wherein the dexamethasone has a particle size distribution with D50 less than 7 μm.

8. The immediate-release solid oral pharmaceutical tablet composition according to claim 1, wherein dexamethasone is present in free base form.

9. The immediate-release solid oral pharmaceutical tablet composition according to claim 1, wherein the composition releases more than 80% of the drug within 30 minutes in 500 ml of 0.1N Hydrochloric acid, using USP I apparatus (Basket) at a temperature of 37±0.5° C. and a rotation speed of 100 revolutions per minute.

10. The immediate-release solid oral pharmaceutical tablet composition according to claim 1, wherein the composition comprises 12.5% by weight of dexamethasone.

11. The immediate-release solid oral pharmaceutical tablet composition according to claim 1, wherein the composition is free from any binder selected from the group consisting of hydroxypropyl methylcellulose, povidone, starch, and microcrystalline cellulose.

12. The immediate-release solid oral pharmaceutical tablet composition according to claim 1, wherein tablet composition, when administered to a human, is bioequivalent within the 80%-125% confidence interval and with a statistical power of at least 80% to a 20 mg tablet of dexamethasone in a bioavailability study in humans.

13. The immediate-release solid oral pharmaceutical tablet composition according to claim 1, wherein the dexamethasone and disintegrant are used in the ratio of from about 1:0.01 to 1:10.

14. The immediate-release solid oral pharmaceutical tablet composition according to claim 1. wherein the dexamethasone and glidant are used in the ratio of from about 1:0.01 to 1:2.

15. The immediate-release solid oral pharmaceutical tablet composition according to claim 1, wherein the dexamethasone and disintegrant are used in the ratio of from about 1:0.1 to 1:4.

16. An immediate-release solid oral pharmaceutical uncoated tablet composition, comprising:
a) dexamethasone present from about 11.25% to 13.5% by weight;
b) from about 35% to about 90% by weight of one or more diluents selected from the group consisting of lactose, mannitol, microcrystalline cellulose, and combinations thereof;
c) from about 0.01% to about 50% by weight of one or more disintegrants selected from the group consisting of croscarmellose sodium, sodium starch glycolate, pregelatinized starch, and combinations thereof;
d) from about 0.01% to about 1% by weight of one or more glidants selected from the group consisting of talc, colloidal silicon dioxide, and combinations thereof; and
e) from about 0.01% to about 2% by weight of one or more lubricants selected from the group consisting of stearic acid, magnesium stearate, and combinations thereof;
wherein the composition is free of any binder selected from the group consisting of hydroxypropyl methylcellulose, povidone, starch, and microcrystalline cellulose, and the composition releases more than 80% of drug within 30 minutes in 500 ml of 0.1N Hydrochloric acid, using USP I apparatus (Basket) at a temperature of 37±0.5° C. and a rotation speed of 100 revolutions per minute and wherein the composition comprises less than 0.5% of 16α-methyl prednisone impurity.

17. The immediate-release solid oral pharmaceutical uncoated tablet composition according to claim 16, wherein tablet composition is prepared by the following process: a) blending a mixture of dexamethasone and at least one pharmaceutically acceptable excipient; b) optionally compacting the blended material using roller compactor; c) optionally milling the compacted material; d) mixing the blend from step a) or milled material from step c) and lubricating with a suitable lubricant; and e) compressing the lubricated blend from step d) into tablets with suitable tooling.

18. An immediate-release solid oral pharmaceutical uncoated tablet composition consisting of:
a) about 11.25% to 13.12% by weight of dexamethasone;
b) about 35% to about 85% by weight of lactose;
c) about 2% to 47% by weight of croscarmellose sodium or pregelatinized starch;
d) about 0.01% to about 0.5% by weight of colloidal silicon dioxide; and
e) about 1% by weight of magnesium stearate;
wherein the composition is free of any binder selected from the group consisting of hydroxypropyl methylcellulose, povidone, starch, and microcrystalline cellulose and the composition comprises less than 1% of total impurities selected from the group consisting of 16α-methyl prednisone, betamethasone, dexamethasone 7,9-diene, desoximetasone and dexamethasone acetate.

19. An immediate-release solid oral pharmaceutical uncoated tablet composition according to claim 18, consisting of:
a) about 12.5% by weight of dexamethasone;
b) about 39% by weight of lactose;
c) about 47% by weight of pregelatinized starch;
d) about 0.25% by weight of colloidal silicon dioxide; and
e) about 1% by weight of magnesium stearate.

20. An immediate-release solid oral pharmaceutical uncoated tablet composition according to claim 18, consisting of:
a) about 12.5% by weight of dexamethasone;
b) about 82% by weight of lactose;
c) about 2% by weight of croscarmellose sodium;
d) about 2% by weight of poloxamer;
e) about 0.25% by weight of colloidal silicon dioxide; and
f) about 1% by weight of magnesium stearate.
